(19) United States
(12) Patent Application Publication
(10) Pub. No.: US 2024/0173316 A1
(43) Pub. Date: May 30, 2024

(54) Methods and Compositions for Treating Cancer

(71) Applicant: Ohio Northern University, Ada, OH (US)

(72) Inventors: Tarek M. MAHFOUZ, Ada, OH (US); Adam Scott BAIR, Worthington, OH (US)

(21) Appl. No.: 18/510,911

(22) Filed: Nov. 16, 2023

Related U.S. Application Data
(60) Provisional application No. 63/425,790, filed on Nov. 16, 2022.

(51) Int. Cl. A61K 31/496; A61K 31/522; A61K 31/5377; A61K 31/708; A61P 35/00; A61P 35/02

(52) U.S. Cl. CPC A61K 31/496; A61K 31/522; A61K 31/5377; A61K 31/708; A61P 35/00; A61P 35/02

(57) ABSTRACT

Described are methods and compositions for inhibiting the interaction between an RGS2 protein and a Gₐₗₚₕₐ protein in a cell. Also described are methods and pharmaceutical compositions of treating cancer in a cell comprising administering to a subject a therapeutically effective amount of an RGS2 inhibitor effective at treating the cancer.

### RELATED APPLICATION

The present claims priority to U.S. Ser. No. 63/425,790 filed Nov. 16, 2022, the disclosure of which is hereby incorporated by reference in its entirety.

### FIELD OF THE DISCLOSURE

The present disclosure relates generally to methods and compositions for inhibiting interaction between an RGS protein and a Gₐₗₚₕₐ protein and, more particularly, to methods and compositions for inhibiting the growth and metastasis of cancer cells.

### BACKGROUND OF THE DISCLOSURE

G-protein Coupled Receptors (GPCR) are the largest family of cell surface receptors in eukaryotes. They allow cells to respond to different types of stimuli such as neurotransmitters, hormones, odorants, taste ligands², and light, by mediating extracellular messages to intracellular signaling pathways. G-proteins are membrane proteins that directly bind to GPCRs' intracellular domains and aid in signal transduction. G-proteins are described as heterotrimeric because they are made up of three different protein subunits named alpha (Gₐₗₚₕₐ), beta (G_{beta}), and gamma (G_{gamma}). G-proteins are classified according to their alpha subunits into four main families; Gₐₗₚₕₐ₋ₛ, Gₐₗₚₕₐ₋ᵢ, G_{alpha-q}, and G_{alpha-12/13}. Agonist binding to GPCRs activates G-proteins causing the Gₐₗₚₕₐ subunit to bind Guanosine Triphosphate (GTP) and dissociate from the heterotrimeric complex to interact with different effectors as follows: Gₐₗₚₕₐ₋ₛ activates adenylyl cyclase, Gₐₗₚₕₐ₋ᵢ inhibits adenylyl cyclase, G_{alpha-q} activates phospholipase C, and the G_{alpha-12/13} regulates small GTP binding proteins. These different Gₐₗₚₕₐ-effector interactions lead to different cell responses and are followed by the alpha subunits hydrolyzing their bound GTP to GDP deactivating themselves and allowing the heterotrimeric complex to reassemble which terminates the GPCR signal. The duration of a GPCR signal is controlled, therefore, by the time it takes a Gₐₗₚₕₐ subunit to hydrolyze its GTP.

RGS is a family of approximately 30 proteins that accelerate the rate at which a Gₐₗₚₕₐ subunit hydrolyzes its GTP and terminates GPCR signaling. They have received special attention in literature because of their ability to modulate GPCR signaling involved in fundamental cellular processes as well as in the physiology of many organs in the human body; in particular the brain and the cardiovascular system. Indeed, the RGS proteins have been implicated in several human diseases such as hypertension, diabetes, depression, and others, and many RGS proteins have been investigated as drug targets. Members of the RGS family share a highly conserved sequence of approximately 120 amino acids, the RGS domain, that mediates direct binding to their Gₐₗₚₕₐ substrates. Binding of the RGS domain stabilizes the flexible switch regions of Gₐₗₚₕₐ which stimulates its GTP hydrolyzing activity. This illustrates the role RGS domain—Gₐₗₚₕₐ switch region interactions play in regulating GPCR signaling and highlights its significance for drug design.

The RGS proteins are divided into eight families based on the sequence similarities of the RGS domain and on the overall protein architecture. Four of those families form the canonical RGSs and the remaining four are non-canonical signaling regulator proteins that have distantly related RGS domains. The canonical RGSs include 20 proteins that are divided into four families (R4, R7, R12, and RZ) with varied Gₐₗₚₕₐ specificities. The R4 family members are known for their promiscuity having the ability to accelerate GTP hydrolysis by both Gₐₗₚₕₐ₋ᵢ and G_{alpha-q}. The exception to this is RGS2, an R4 member that shows specificity to G_{alpha-q}; at least in vitro. RGS2 has been shown to be overexpressed in human breast cancer cells and in metastatic prostate cancer, which suggests a role for RGS2 in tumor progression and metastasis. Indeed, it has been shown that the overexpression of RGS2 in the LNCaP prostate cancer cell line increased the cells' migrating ability whereas knock-down of RGS2 inhibited migration ability. Moreover, there is a statistically significant correlation between RGS2 levels in samples taken from cancer patients and cancer specific patient survival. In addition, knock-down of the G_{alpha-q} subunit promotes invasiveness and metastatic properties of lung cancer cell lines. This would be similar to the overexpression of RGS2 since RGS2 is an inhibitor to G_{alpha-q}. Inhibitors of RGS2 function, therefore, might add to our arsenal of chemotherapeutic agents. So far, however, no RGS2 inhibitors have been developed or tested for their anticancer activity.

### SUMMARY OF THE DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a method of treating a subject with a cancer that includes administering to the subject an RGS2 inhibitor in an amount therapeutically effective to treat the cancer. The RGS2 inhibitor interacts with the RGS2 protein to sterically hinder its interaction with a Gₐₗₚₕₐ protein. In embodiments, the cancer may be a solid cancer or a blood cancer. In embodiments, the cancer may include a breast cancer, a prostate cancer, a renal cancer, an ovarian cancer, a melanoma, a colon cancer, a non-small cell lung cancer, a leukemia, a central nervous system cancer. In embodiments, the RGS2 inhibitor may be administered via oral administration, injection administration, or combinations thereof. In embodiments, the injection administration may include injection administration is selected from the group consisting of intravenous injection, intra-arterial injection, subcutaneous, intramuscular injection, peritoneal injection, intrathecal injection, or combinations thereof.

In another embodiment, a pharmaceutical composition is described that includes an RGS2 inhibitor in an amount effective to inhibit the interaction between an RGS2 protein and a Gₐₗₚₕₐ protein, which include a G_{alpha-q} protein, in a cancer cell in a subject. In embodiments, the pharmaceutical composition include an RGS2 inhibitor formulated for administration to a subject via oral administration, injection administration, or combinations thereof.

In a further embodiment, method of inhibiting the interaction between an RGS2 protein and a Gₐₗₚₕₐ protein, which include a G_{alpha-q} protein, in a cell is described that includes administering to the cell an RGS2 inhibitor in an amount capable of non-covalently interacting with the RGS2 protein. In embodiments, the cell is a cancer cell.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a computer model showing portions of the ribbon structures for RGS2 and Gₐₗₚₕₐ proteins and the targeted pharmacophore.

FIG. 2 is a computer model showing portions of the ribbon structures for RGS2 and Gₐₗₚₕₐ proteins showing an RGS2 inhibitor AJ-1 docked to the targeted pharmacophore.

FIG. 3A is a photomicrograph comparing the effects of an RGS2 inhibitor (1 μM AJ-3) with a control (DMSO) on a scratch assay after 24 hours.

FIG. 3B is a series of photomicrographs comparing the effects of an RGS2 inhibitor (1 μM AJ-3) with a control (DMSO) on a scratch assay after 4 days (top panels) and 5 days (bottom panels).

FIG. 3C is a photomicrograph comparing the effects of an RGS2 inhibitor (2 μM AJ-3) with a control (DMSO) on a scratch assay after 24 hours.

FIG. 3D is a series of photomicrographs comparing the effects of an RGS2 inhibitor (2 μM AJ-3) with a control (DMSO) on a scratch assay after 4 days (top panels) and 5 days (bottom panels).

FIG. 3E is a photomicrograph comparing the effects of an RGS2 inhibitor (5 μM AJ-3) with a control (DMSO) on a scratch assay after 24 hours.

FIG. 3F are a series of photomicrographs comparing the effects of an RGS2 inhibitor (5 μM AJ-3) with a control (DMSO) on a scratch assay after 4 days (top panels) and 5 days (bottom panels).

FIG. 3G is a photomicrograph comparing the effects of an RGS2 inhibitor (10 μM AJ-3) with a control (DMSO) on a scratch assay after 24 hours.

FIG. 3H are a series of photomicrographs comparing the effects of an RGS2 inhibitor (10 μM AJ-3) with a control (DMSO) on a scratch assay after 4 days (top panels) and 5 days (bottom panels).

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally to methods and compositions for treating cancer and, more particularly, to methods and compositions for killing, inhibiting the growth, and inhibiting metastasis of cancer cells.

Described herein are the first direct selective inhibitors of RGS2 protein family employed as a cancer therapeutic. The novelty of this mechanism lends an inherent lack of therapeutic resistance from cancer cells, and its simplicity is an appealing alternative to the more complicated chemotherapies available.

GPCRs are cell surface receptor proteins involved in transforming extracellular messages into internal cellular signaling. In particular, these G-protein signals have the ability to inhibit cancer progression. A portion of the G-protein, a subunit called Gₐₗₚₕₐ, has a protein family known as G_{alpha-q} that has been linked to metastasis. To activate its signal, Gₐₗₚₕₐ binds Guanosine Triphosphate (GTP) which causes Gₐₗₚₕₐ subunit to dissociate from the receptor subunit and activate its signal. The Gₐₗₚₕₐ subunit then undergoes a self-deactivation mechanism via hydrolysis of said GTP to GDP to turn off the G-protein signal. It has been observed that an abnormal loss of the Gₐₗₚₕₐ signal leads to aggressive growth and migration of cancerous cells. The RGS protein family functions as an inhibitor of Gₐₗₚₕₐ, accelerating the rate of GTP hydrolysis to turn off G-protein signaling.

RGS2, a member of the R4 family, is a specific inhibitor of G_{alpha-q}, and high levels of RGS2 protein in cancer patients correlate with poor survival rates and high metastatic status. In separate studies, genetic knock downs of RGS2 have exhibited a notable decrease in cancer cell progression. The RGS2 protein shares conserved amino acid domains that interact with G_{alpha-q} that serve as suitable targets for inhibitory drug design. Utilizing a structure-based pharmacophore model targeting these interaction domains, ten RGS2 inhibitors (i.e., AJ-1, AJ-2, AJ-3, AJ-4, AJ-5, AJ-6, AJ-7, AJ-8, AJ-9, and AJ-10, structures illustrated in Table 1) with diverse scaffolds exhibiting competency in non-covalently binding the RGS2 proteins were discovered. As illustrated in the examples below, these compounds non-covalently bind to RGS2, and exhibited anti-cancer properties against a plethora of cancer types.

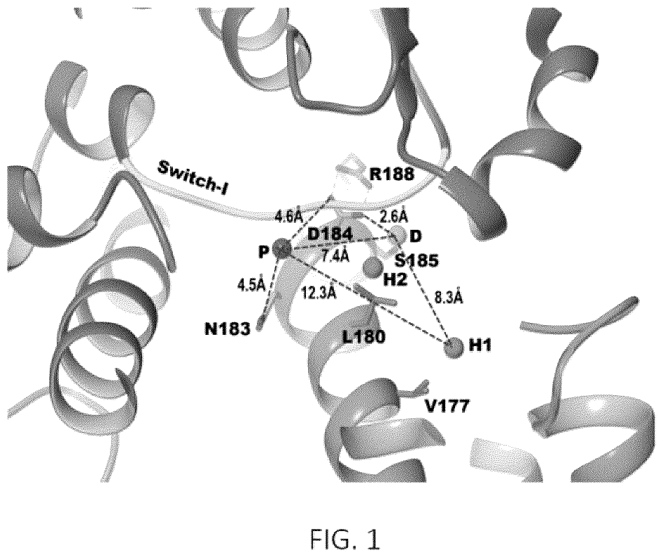

Without being bound to a particular theory, embodiments of the RGS2 inhibitors described herein interact with a pharmacophore to interrupt the interaction between RGS2 and its target G_{alpha-q} protein, allowing an inhibitory signal from the G protein receptor to subdue the growth and metastasis of aggressive cancer cells. In embodiments, the RGS2 inhibitor non-covalently may interact with a pharmacophore defined by turn-183 of human RGS2. The pharmacophore may be further defined by a first asparagine at residue 183 (“N183”), a second asparagine at residue 184 (“N184”), and a serine at residue 185 (“S185”). N183 and N184 may interact with a positively charged pharmacophore site (P) and S185 may interact with a hydrogen-bond donor pharmacophore site (D) within the RGS2 inhibitor pharmacophore. The P and D sites may be about 7.4 Å apart and provide RGS2 selectivity for inhibitors. In embodiments, the pharmacophore may include two hydrophobic sites (H1 and H2) that may help stabilize the interaction of an inhibitor in the correct orientation. Site H1 is at a distance of about 3.1 Å from the side chains of valine at residue 177 (“V177”), and H2 is at a distance of about 3.4 Å from the side chain of leucine at residue (“L180”). H1 is about 12.3 Å from site P and about 8.3 Å from site D, FIG. 1, so that identified compounds may cover the turn-183 surface and inhibit the interaction between RGS2 and a Gₐₗₚₕₐ protein.

In embodiments, cancer may include solid cancers, lymphomas, and leukemias. Examples of different types of cancers include, but are not limited to, prostate cancer, lung cancer (e.g., non-small cell lung cancer or NSCLC), ovarian cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma, head and neck cancer, blood cancer, endometrial cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma. In some instances, the cancer can be metastatic. In certain instances, the cancer is prostate cancer, lung cancer, breast cancer, liver cancer, ovarian cancer, endometrial cancer, bladder cancer, colon cancer, gastric cancer, lymphoma, or a glioma such as glioblastoma multiforme.

In embodiments, when treating cancer, which includes slowing the growth of cancer tissue, decreasing cancerous tumor size, killing cancer cells, and decreasing metastasis, a therapeutically effective dose of at least one RGS2 inhibitor is administered to a subject having cancer. The therapeutically effective dose of at least one RGS2 inhibitor is a dose that achieves levels of RGS2 inhibitor, and its active metabolites, at the site of the cancer to result in treating the cancer. In embodiments, success in treating the cancer may be measured using standard clinical methodologies capable of, for example, detecting decreases in rate of cancer growth, decreases in rate of cancer spread, decreases in cancer size, decreases in the number of cancerous lesions, or combinations thereof.

In embodiments, the at least one RGS2 inhibitor may be administered with one or more other anti-cancer therapeutic agents as a combination therapy or as a secondary treatment. Other anti-cancer therapeutic agents are known in the art.

In embodiments, the at least one RGS2 inhibitor may be administered by any route of administration that results in the therapeutically effective dose of the at least one RGS2 inhibitor, or its active metabolites, at the site of the cancer. One of ordinary skill will appreciate that the route of administration may vary based on the health of the patient, the type of cancer being treated, the location of the cancer being treated, as well as other factors such as clearance rates for the at least one RGS2 inhibitor and to limit potential side effects.

In an embodiment, the therapeutically effective dose of the at least one RGS2 inhibitor may be administered via oral administration. The dose of the at least one RGS2 inhibitor may be administered in a single dose or multiple doses in a 24 hour period and may generally be administered for a specified period of time such as for about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31 or more days.

Embodiments of the disclosure are directed to a method of treating cancer in a subject comprising administering via injection a therapeutically effective amount of at least one RGS2 inhibitor. In embodiments, the injection may include one or more of intravenous injection, intra-arterial injection, subcutaneous, intramuscular injection, peritoneal injection, or intrathecal injection. In embodiments, the injection is into or near the site or sites of the cancer.

In embodiments, the therapeutically effective dose of the at least one RGS2 inhibitor administered in a bolus injection, by continuous infusion, or a combination of bolus injection and continuous infusion. The term bolus injection is understood to be an injection wherein the dose is delivered over a relatively short period of time. The term continuous infusion is understood to be an injection delivered, such as with an intravenous drip, wherein the dose is delivered in a metered manner over the period of time desired for the anticancer therapy. In an embodiment, therapeutically effective amount of the at least one RGS2 inhibitor may be administered via continuous infusion over a period of time ranging between about 30 min/day to about 24 hours/day. In another embodiment, therapeutically effective amount of the at least one RGS2 inhibitor may be administered over a period of about 8 hours/day to about 24 hours/day. In some circumstances, the therapeutically effective amount of the at least one RGS2 inhibitor may be administered via continuous drip on at least one day and up to on seven days. In other embodiments, the therapeutically effective amount of the at least one RGS2 inhibitor may be administered for even longer periods of times, such as on days over multiple weeks, months, or even years, as necessary to treat the cancer in the subject. Thus, embodiments of the invention are directed to the long term administration of the therapeutically effective amount of the at least one RGS2 inhibitor to treat cancer in the subject.

In some instances a combination of bolus injection with continuous infusion may be desired to treat cancer in a subject. For example, a bolus injection may be utilized to deliver a loading dose, i.e., a dose of the at least one RGS2 inhibitor to rapidly achieve a desired therapeutic level of the at least one RGS2 inhibitor in the subject, and the continuous infusion may be utilized to maintain or even titrate the desired therapeutic levels over the desired duration of treatment.

In embodiments, the RGS2 inhibitor has a half-maximal inhibitory concentration (IC50) value of from about 100 nM to about 100 μM, e.g., from about 100 nM to about 50 μM from about 100 nM to about 25 μM, from about 100 nM to about 10 μM. from about 500 nM to about 100 μM, from about 500 nM to about 50 μM, from about 500 nM to about 25 μM, from about 500 nM to about 10 μM, from about 1 μM to about 100 AM, from about 1 μM to about 50 μM, from about 1 μM to about 25 μM, from about 1 μM to about 10 μM, or about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM. In some instances, the IC50 value for a specific compound of RGS2 inhibitor, such as AJ-1, AJ-2, AJ-3, AJ-4, AJ-5, AJ-6, AJ-7, AJ-8, AJ-9, and AJ-10, is measured using an in vitro assay in cancer cells that have been incubated with the compound. The IC50 value can be determined based on the effect of the compound in inhibiting the survival of cancer cells such as cells from a cancer cell line or primary tumor cells. In other embodiments, the RGS inhibitor has an inhibitor constant (Ki) that is essentially the same numerical value as the IC50 value, or is about one-half the value of the IC50 value.

RGS2 inhibitor, as used herein, includes compositions that interrupt the interaction between RGS2 and its target G protein receptor to subdue the growth or metastasis of cancer cells. In embodiments, the RGS2 inhibitor may include AJ-1, AJ-2, AJ-3, AJ-4, AJ-5, AJ-6, AJ-7, AJ-8, AJ-9, or AJ-10, as described herein, or pharmaceutically acceptable salts or solvates of these RGS2 inhibitors, as well as, pro-drugs, isomers, and polymorphs, as well as derivatives that are structurally related to or derived therefrom that function as a RGS2 inhibitor. The compositions of RGS2 inhibitors can be administered in vivo in a pharmaceutically acceptable carrier. By “pharmaceutically acceptable” is meant a material that is not biologically or otherwise undesirable. Thus, the pharmaceutically acceptable carriers may be administered to a subject without causing undesirable biological effects or interacting in a deleterious manner with any of the other components of the pharmaceutical composition in which it is contained. The carrier may be selected to minimize any degradation of the RGS2 inhibitors and to minimize any adverse side effects in the subject, as would be known to one of skill in the art.

Suitable carriers and their formulations are described in Remington: The Science and Practice of Pharmacy (19th ed.) ed. A. R. Gennaro, Mack Publishing Company, Easton, Pa. 1995. For intravenous administration, an appropriate amount of a pharmaceutically-acceptable salt may be used in the formulation to render the formulation isotonic. Examples of the pharmaceutically-acceptable carriers include, but are not limited to, saline, Ringer's solution and dextrose solution. The pH of the solution is in a pharmaceutically acceptable range, preferably from about 5 to about 8.5, and more preferably from about 7.8 to about 8.2. Further carriers include sustained release preparations such as semipermeable matrices of solid hydrophobic polymers containing the pharmaceutical composition, which matrices are in the form of shaped articles, e.g., films, liposomes or microparticles. It will be apparent to those persons skilled in the art that certain carriers may be more preferable depending upon, for instance, the route of administration and concentration of composition being administered. For example, persons skilled in the art may choose a particular carrier suitable for introduction to the body by injection, as described above, or ingestion.

For ingestion, the RGS2 inhibitor may be formed into a tablet, capsulized, or dissolved or suspended in a liquid or gel as known to those of ordinary skill in the art for oral administration of a drug. In some embodiments, the RGS2 inhibitor is formulated for sustained release, such as with the use of one or more excipients that control the release of the RGS2 inhibitor over a specified period of time for absorption by the subject.

The pharmaceutical compositions of RGS2 inhibitors may also include binders, thickeners, diluents, buffers, preservatives, surface active agents, and the like in addition to the RGS2 inhibitors and carriers.

The disclosed compositions may be suitable for systemic administration or localized administration. For example the compositions may be administered by other means known in the art, such as orally, parenterally (e.g., intravenous injection, intramuscular injection, intraperitoneal injection, intrathecal, or subcutaneous injection), suppository, or even transdermally such as through a gel or patch formulation. Such formulations may be prepared as is known to those of ordinary skill in the art.

### EXAMPLE 1

A study was performed to identify candidate pharmaceutical formulations with simultaneous high binding affinity and selectivity to RGS2 that would perform an inhibitory function on the RGS2-G_{alpha-q} protein-protein interaction.

### Hardware and Software

Pharmacophore building and database searching were performed on an Ibex 4260 LINUX system with an Intel Pentium-D 2.8 GHz dual core processor and 1 GB RAM with UBUNTU 14.04 operating system. PHASE, as implemented in Schrodinger release 2014-2³¹, was used for pharmacophore modeling and database searching. All docking calculations were performed on a PC with Windows 8 operating system. AUTODOCK VINA (VINA) was used for docking of hits. CHIMERA was used for visualization and examination of docking results and ranking. Both CHIMERA and VMD were used to prepare the associated figures.

### Structures Used

The crystal structure of the SDK triple mutant the human RGS2 in complex with a transition state mimetic form of the Gₐₗₚₕₐ₋ᵢ subunit (PDB code 2V4Z) was used to identify pharmacophore sites and positioning of sites. Crystal structure 2AF0 of the free human RGS2 was used to identify structures with good binding potential. Structures were docked to RGS1; chain D of structure 2GTP. Inhibitors were also docked to the following human RGSs: RGS3 (PDB code 2OJ4), RGS8 (PDB code 2IHD), and RGS18 (PDB code 2OWI).

### Feature Type and Coordinates of Pharmacophore Sites

For each RGS2-Gₐₗₚₕₐ₋ᵢ interacting pair of residues in structure 2V4Z, all terminal side chain atoms from the two proteins that are within a distance of 5 Å from each other were combined into one VMD selection. The center of mass for the selection was calculated using VMD and the xyz coordinates for the center of mass served as the coordinates for the corresponding pharmacophore site relevant for PHASE. The type of pharmacophore feature at each site complemented the chemical group in the RGS2 amino acid side chain as explained below.

### Database Construction and Screening

The ZINC database collections were downloaded in sdf format and converted

to three dimensional structures using PHASE. The structures were 1) neutralized, 2) minimized for 100 steps retaining its specified chirality, and 3) pharmacophore sites were generated. Conformers were then generated starting from the minimized structures using the “thorough sampling” method with 100 steps per rotatable bond. A maximum of 1,000 conformers per structure was specified. All conformers that differed from the lowest energy conformer by more than 70 kcal/mol were rejected. A 1.0 Å RMSD cutoff for redundant conformers was implemented. To evaluate conformational energies, the OPLS_2005 force field and the GB/SA solvation treatment were used as implemented in PHASE. All generated conformers were post-minimized for 50 steps.

### Docking

To facilitate comparison of inhibitor docking to the different RGS structures, all RGS structures used in this research were superimposed on RGS1 (chain D in structure 2GTP), using CHIMERA's MATCHMAKER utility, before defining the search space for docking. The search space was then defined by creating a box centered at x=1.06, y=48.20, z=17.31 and with xyz dimensions of x=50.88, y=34.31, z=31.08 using CHIMERA's VINA utility. These dimensions ensured that all protein atoms from the different RGS structures are part of the search space to help identify areas in RGS structures where inhibitors prefer to bind. The same box was used in docking the different RGS structures. For batch docking of inhibitors using VINA, PDBQT files were generated for RGSs and inhibitors. To prepare protein PDBQT files, each of the superimposed RGS structure was saved in its transformed coordinates to a separate PDB file. PDB files were then imported one at a time into AUTODOCK MGL TOOLS where polar hydrogens were added and the final structure was saved in PDBQT format. PDBQT files for inhibitors were generated in batch mode using command line prompts where rotatable bonds were defined and Gasteiger charges were added and the final structures were saved in PDBQT format. Configuration files for batch docking contained the box information indicated above and the following docking parameters: the seed option was set to 1000, the energy_range to 7, the exhaustiveness to 10, and the num_modes to 100. Batch docking was started by invoking VINA from the command line.

### NanoBit RGS/Gα Cell-Based Protein-Protein Interaction Assay

The NanoBit cell-based assay was performed as previously described. The NanoBit substrate (NanoGlo Live Cell Reagent) was prepared as a 5× stock, per the manufacture's protocol, and added to the wells at 10 μL per well. Post substrate addition, a baseline signal was established for 30 minutes using a BioTek Synergy 2 plate reader in luminescent mode. Post baseline read 10 μL of vehicle (Hank's Balanced Salt solution+20 mM HEPES) or AlF₄ (stock solution: 40 mM NaF, 500 μM AlCl₃) was added and luminescence was measured for an additional 30 minutes. Lastly, 4× compound or vehicle (DMSO) was added to the appropriate wells, and the plate was read for an additional hour. Data presented represents the mean of N=3 independent experiments±S.E.M. All data analysis was completed in GraphPad Prism 7 and 8.

### Differential Scanning Fluoremetry (DSF)

Solutions of RGS2 (or G_{αq}) with and without inhibitors were aliquoted into the wells of a 96-well plate and were incubated at room temperature for 30 min. After the addition of 2× Sypro Orange dye, fluorescence was measured from −20-80° C. using a Roche 480 LightCycler II. Melting Temperatures were calculated using DMAN.

### Anticancer Activity

To determine IC₅₀ values, MCF-7 cells (in phenol red free media and 10% FBS) were plated into a 96 well plate at 5000 cells/ml, 100 μL/well, and allowed to attach overnight. The test compound was added to each row in a serial dilution giving 8 concentrations (and 8 duplications/concentration). Additional wells contained control (nothing added) and vehicle control (vehicle only). After 48 hours, the media was removed, and was replaced with 100 ml of phenol red free media that contained 0.4 mg/ml XTT according to the manufacturer's instruction (Sigma-Aldrich). After 4 hours, absorption at 450 nm was measured, and data are expressed as % growth compared to control. IC₅₀ is determined using the statistics program PRIZM.

### NMR Experiments

Proton NMR was performed to verify purchased compounds' structures and purity. Approximately, 5 mg of inhibitor was dissolved in ˜0.5 mL of dimethyl sulfoxide-d₆. The solution was filtered and transferred to a disposable 5 mm NMR tube. Proton NMR spectra were collected using a 400 MHz Bruker spectrometer. The acquired spectra were compared to supplier's provided data.

### RGS2 Pharmacophore Modeling

In embodiments of the RGS2 inhibitor, interactions with the Gₐₗₚₕₐ switch regions are essential for binding the RGS proteins. Structure 2V4Z shows the side chains of N183, D184, and S185 of RGS2 (hereinafter “turn-183”) form direct contacts with the Gₐₗₚₕₐ₋ᵢ switch-I region, FIG. 1. To inhibit RGS2 function, therefore, the present pharmacophore model targeted turn-183 of the human RGS2 to sterically occlude the switch-I interaction surface of its RGS domain. A challenge to consider was selectivity towards RGS2. Selectivity is a challenge due to the conserved nature of the RGS domain that would allow inhibitor binding to multiple RGS proteins in particular members of the R4 family to which RGS2 belongs. To enhance inhibitor selectivity, the model must include sites that target features and residues unique to RGS2 among the other RGSs, especially the R4 family members. In this regard, of the three residues forming turn-183, residue 183 is asparagine in RGS2, but it is conserved lysine in the closely related R4 RGSs. Residue 184 in RGS2 is also asparagine; but it is conserved aspartic acid in all other RGSs including the R4 family. In the other RGSs, this particular residue, D184, forms a salt bridge with the side chain of R188, which brings another positive charge (from the arginine side chain) to turn-183 region, as shown in FIG. 1. This is not the case with RGS2 due to the neutral asparagine side chain at this position. A positively charged (P) pharmacophore site may be located between the side chains of N183 and N184, and a hydrogen-bond donor (D) pharmacophore site may be located in between the side chains of N184 and S185, FIG. 1, which enhanced selectivity towards RGS2. This resulted in positively charged and donor groups of inhibitors forming favorable dipole-dipole or hydrogen bonding interactions with turn-183 of RGS2, but being repelled by the positive charges on turn-183 in the other RGSs. The P and D sites were 7.4 Å apart, which spans the entire contact area with the Gₐₗₚₕₐ switch-I region, FIG. 1.

The P and D site, therefore, are responsible for selectivity. However, on their own, the interactions they form may not be enough to stabilize inhibitors in place or ensure proper orientation. Two hydrophobic sites (H1 and H2) overcome this deficiency. Site H1 is at a distance of 3.1 Å from the side chains of V177, and H2 is at a distance of 3.4 Å from the side chain of L180. These two sites provide favorable interactions to further stabilize inhibitor binding, to provide more specificity in binding to turn-183, and to ensure proper orientation of inhibitors. These two sites are hydrophobic due to the hydrophobic nature of this region, leucine and valine side chains. H1 is 12.3 Å from site P and 8.3 Å from site D, FIG. 1, so that identified compounds would be big enough to cover the turn-183 surface.

Database searching and estimation of binding affinity—The pharmacophore model was used to query to search different collections of the ZINC database (https://zinc.docking.org/). In searching databases, molecules mapped to at least three of the four pharmacophore sites as follows: molecules mapped to sites P and D to ensure selectivity towards RGS2 as well as maximal coverage of turn-183 surface. In addition, molecules matched to at least one of the two H sites to maximize the number of hits obtained while maintaining minimum level of binding specificity and proper orientation in the binding site

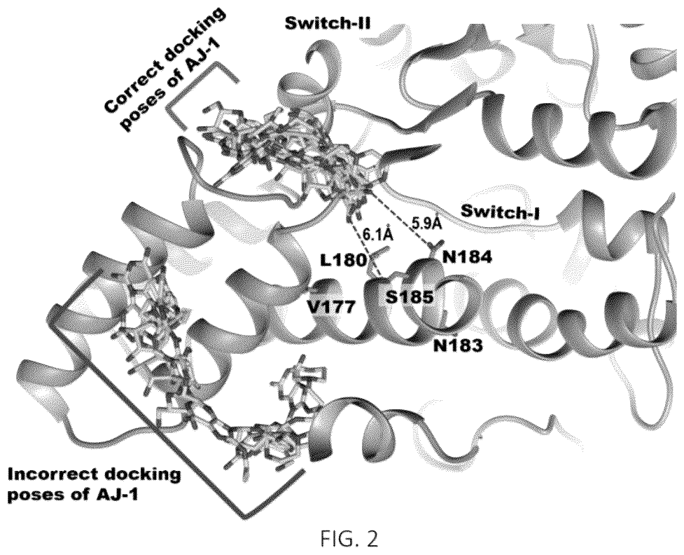

Not all retrieved hits inhibited RGS2 function because matching molecules, despite having the groups required for inhibition, also have other functional groups that may cause them to bind different from expected. All retrieved hits were batch docked to RGS2 using VINA to ensure molecules showed correct docking poses. A correct docking pose is defined here as a pose where the docked molecule binds within about 6 Å of N183 N184, or S185 with a docking score of not less than −5.0. Structure 2AF0 was used for this docking step because it contains the free-unbound wild type human RGS2 and provided more relevant results. Molecules were allowed to bind anywhere on RGS2 and regions on the protein where the resulting docking poses cluster were noted to identify how likely the molecule would be to bind to turn-183. This was done by creating a docking box big enough to include all protein atoms. Upon completion of docking, all generated poses for each molecule were inspected visually and the number of poses bound to turn-183 were recorded together with their docking scores. Molecules of the present disclosure showed a high number of correct docking poses and/or those with an average docking score of at least −5.0. Inhibitor AJ-1 showed the most number of correct poses that, most importantly, were shown to sterically occlude both switch-I and switch-II regions upon overlaying the G subunit on the docked structures, FIG. 2.

### Selectivity Towards RGS2

Since RGS2 selectivity was a concern, potential binding of identified hits to other RGS proteins was evaluated. This was done through docking, similar to the previous step but it included two steps; first, molecules were docked to human RGS1; chain D of structure 2GTP, and, second, only molecules that showed the greatest RGS2 selectivity were docked to other human RGSs. In both docking steps, selectivity was evaluated by counting the number of correct docking poses generated with RGS2 and comparing that number to that generated with each of the other RGSs for the same molecule. Molecules that generated more correct poses with RGS2 than with RGS1 were considered selective.

TABLE 1
Ten inhibitors with docking and assay results. Inhibitors are listed in order decreasing IC₅₀.

| | | | | RGS2 | RGS4 | |
|---|---|---|---|---|---|---|
| | RGS2 | Docking | RGS1 | IC₅₀ μM | IC₅₀ μM | Select- |
| Structure/Name | Poses | Score | Poses | (95% Cl) | (95% Cl) | ivity¹ |
| 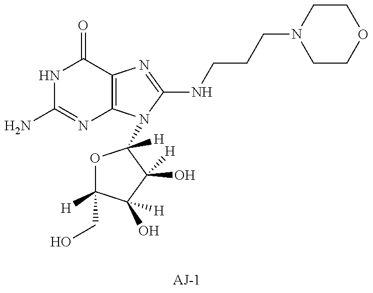 [SMILES: Nc1nc2c(nc(NCCCN3CCOCC3)n2[C@H]2O[C@@H](CO)[C@H](O)[C@@H]2O)c(=O)[nH]1] | 10 | −5.00 | 0 | 0.04 (0.012 to 0.11) | 10.10 (2.1 to 51) | 289 |
| 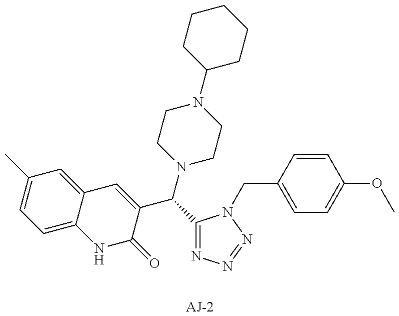 | 4 | −7.00 | 0 | 1.45 (0.34 to 4.5) | 18.80 (8.4 to 44) | 13 |
| 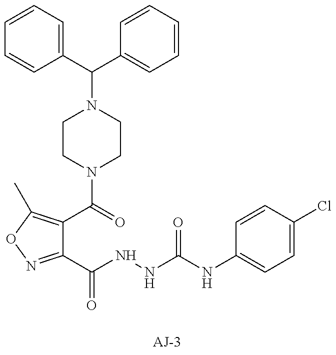 [SMILES: Cc1onc(C(=O)NNC(=O)Nc2ccc(Cl)cc2)c1C(=O)N1CCN(C(c2ccccc2)c2ccccc2)CC1] | 4 | −6.60 | 1 | 4.94 (2.6 to 8.24) | 21.00 (4.4 to 200) | 4.25 |
| 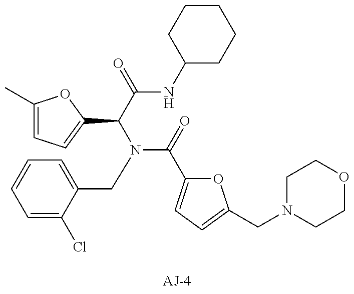 [SMILES: Cc1ccc([C@@H](C(=O)NC2CCCCC2)N(Cc2ccccc2Cl)C(=O)c2ccc(CN3CCOCC3)o2)o1] | 4 | −6.10 | 0 | 13.80 (2 to 106) | 25.00 (12 to 93) | 1.81 |
| 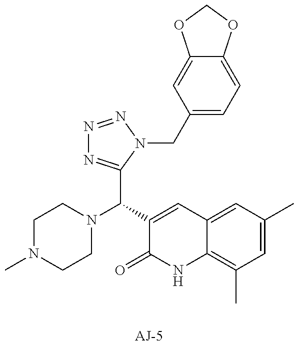 | 2 | −7.30 | 0 | 15.00 (5.2 to 50) | 23.00 (4.0 to 396) | 1.53 |
| 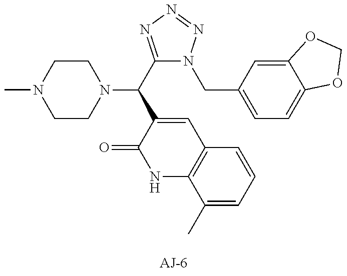 [SMILES: Cc1cccc2cc([C@H](c3nnnn3Cc3ccc4c(c3)OCO4)N3CCN(C)CC3)c(=O)[nH]c12] | 6 | −6.20 | 0 | 19.80 (4.2 to 129) | ~9.60 (wide) | 0.48 |
| 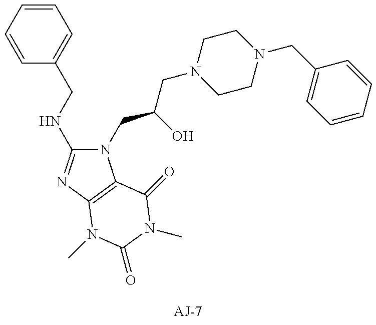 [SMILES: Cn1c(=O)c2c(nc(NCc3ccccc3)n2C[C@H](O)CN2CCN(Cc3ccccc3)CC2)n(C)c1=O] | 5 | −6.30 | 0 | 27.60 (12.1 to 71.5) | 14.10 (4.7 to 44) | 0.51 |
| 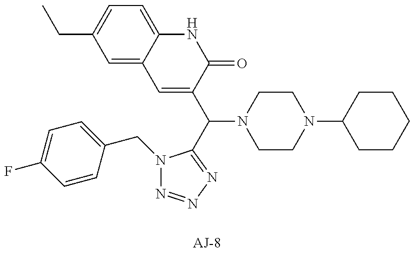 [SMILES: CCc1ccc2[nH]c(=O)c(C(c3nnnn3Cc3ccc(F)cc3)N3CCN(C4CCCCC4)CC3)cc2c1] | 4 | −6.70 | 0 | 33.10 (3.0 to N/A) | 9.60 (4.4 to 22.3) | 0.29 |
| 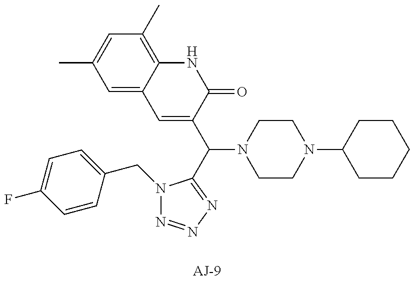 [SMILES: Cc1cc(C)c2[nH]c(=O)c(C(c3nnnn3Cc3ccc(F)cc3)N3CCN(C4CCCCC4)CC3)cc2c1] | 5 | −6.20 | 0 | 33.50 (7.3 to 448) | 91.00 (43 to 221) | 2.72 |
| 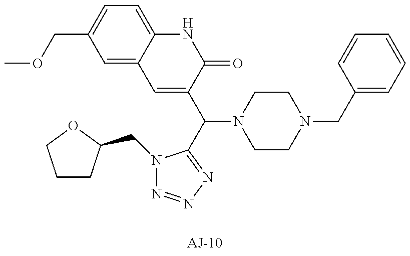 [SMILES: COCc1ccc2[nH]c(=O)c(C(c3nnnn3C[C@H]3CCCO3)N3CCN(Cc4ccccc4)CC3)cc2c1] | 5 | −6.10 | 0 | 78.00 (13.7 to N/A) | N/A | N/A |

¹Selectivity = RGS4 IC₅₀/RGS2 IC₅₀

### RGS2 Inhibition and Selectivity Assays

It is to be noted that all molecules purchased were subjected to H¹-NMR analysis to verify structure and purity. The results matched the structures in Table 1 and showed all analyzed samples to be at least 90% pure.

All selected compounds were purchased and tested for their ability to inhibit RGS2-G_{alpha-q} interaction in NanoBit assays. The same assay was also used to test the compounds' ability to inhibit RGS4-G_{alpha-q} interaction as a measure of selectivity. RGS4 was not part of the docking calculations performed here because no structure is available for the human RGS4. It was used in the assays, nonetheless, to test for the validity of our docking approach to selectivity and binding. As Table 1 shows, all 10 compounds selected inhibited RGS2-G_{alpha-q} interaction with moderate to high potency. AJ-1 proved to be the most potent inhibitor, with an IC₅₀ value of 0.04 μM, which was expected since it showed the largest number of correct poses with RGS2, despite having the lowest average docking score. AJ-1 also proved to be highly selective towards RGS2 over RGS4 with a selectivity ratio of 289; second only to AJ-10.

AJ-10 is a moderate inhibitor with an IC₅₀ of 7804 but it the most selective of all inhibitors tested since we could not determine an IC₅₀ for it with RGS4.

These results show reasonable agreement between our computational predictions and the experimental results. It also illustrates the importance of taking both the number of poses together with the docking scores into account when interpreting docking results instead of relying on one but not the other.

### Anticancer Activity of RGS2 Inhibitors

Several studies have reported the over expression of RGS2 in different cancers especially metastatic ones. If RGS2 truly played a role in cancer development and/or metastasis then inhibitors of its function should have anticancer activities. Nine inhibitors were submitted to the National Cancer Institute NCI-60 program for anti-cancer screening. All inhibitors submitted inhibited the growth of several cancer cell lines with varying potency. In addition, the ability of AJ-3 to inhibit the migration of the LNCaP prostate cancer cell line was tested. AJ-3 inhibited the migration of LNCaP cells at concentrations as low as 1 μM but more potently at 2 μM and higher concentrations.

### Conclusions

Protein-protein interactions are usually challenging to disrupt because the interaction surfaces are usually shallow and featureless. In this disclosure, we present an approach to target protein-protein interactions with high degree of selectivity. Following that approach allowed us to identify inhibitors of RGS2-G_{alpha-q} interactions. In developing the pharmacophore model, we focused on the interaction surface between turn-183 of RGS2 and the G_{alpha-q} switch-I region to discover essential binding information. That interaction surface is important because: 1) turn-183 is part of the RGS domain essential for binding Gₐₗₚₕₐ subunits and the switch-I region is part of the flexible switch region of G_{alpha-q} that is essential for its function. 2) the amino acid sequence of turn-183 has residues unique to RGS2 among the other RGS proteins and these differences could be utilized to enhance selectivity towards RGS2.

The pharmacophore model developed was extremely successful in identifying potent inhibitors of RGS2-G_{alpha-q} interactions and the steps that followed were effective in determining inhibitors selective towards RGS2. Of the identified inhibitors, AJ-1 is the most potent with an IC₅₀ of 0.04 μM and the least potent was AJ-10 with an IC₅₀ of 78 μM. With regards to RGS2 selectivity, AJ-10 is the most selective with no apparent activity towards RGS4 and the second most selective inhibitor is AJ-1 with selectivity ratio (RGS4 IC₅₀/RGS2 IC₅₀) of 289. AJ-6, AJ-7, and AJ-8 are actually more potent towards RGS4 than they are towards RGS2 with selectivity rations of 0.48, 0.51, and 0.29; respectively. The RGS2 inhibitors presented here inhibited the growth of multiple cancer cell lines with varying potency and, additionally, AJ-3 inhibited the migration ability of the prostate cancer LNCaP cells.

The present disclosure indicates the validity of our two-step approach to inhibit protein-protein interactions with high potency and selectivity. The structure-based pharmacophore model allowed us to identify molecules with functional groups necessary for binding and docking allow us to focus on molecules that produce the desired binding modes. Our results also show that, in interpreting docking results, more accurate results can be obtained when we take into account both the number of relevant docking poses and the docking scores instead of relying solely on one but not the other. Finally, our results indicate important role for RGS2 in cancer development and metastasis and show that inhibitors of RGS2 function have a promising potential in cancer chemotherapy.

### EXAMPLE 2

In a separate study, the metastatic potential of RGS2 in an aggressive cancer cell model was investigated. Two separate surveys on human cancer cells was used to determine whether RGS2 loss, and/or a drug compound directly inhibiting RGS2 would impact the metastatic ability of cancer cells.

It has previously been shown that RGS2 gene expression in the human prostate cancer cells LnCap were downgraded by infection with small hairpin RNA (shRNA) targeting the RGS2 gene (henceforth “shRGS2”). These shRGS2 LnCap cells expressed lower levels of RGS2 protein compared to the non-infected control, leading to a reduction in RGS2 activity. Data are presented here that show the effects of treating LnCap cells with a range of dosages from about 1 μM to about 10 μM of RGS2 inhibitor, and growth was observed for a maximum of 120 hours post-treatment.

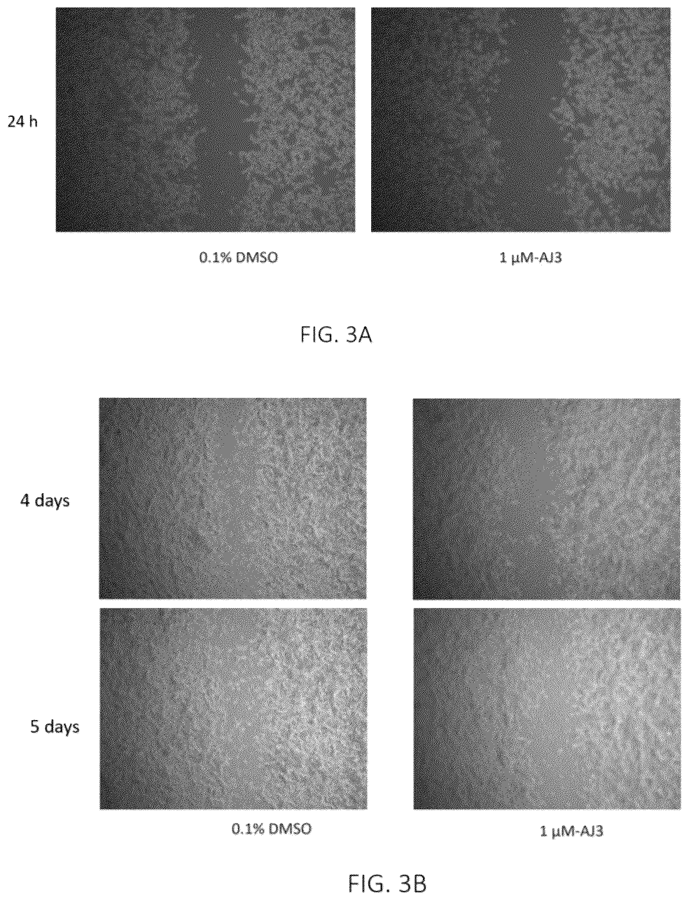

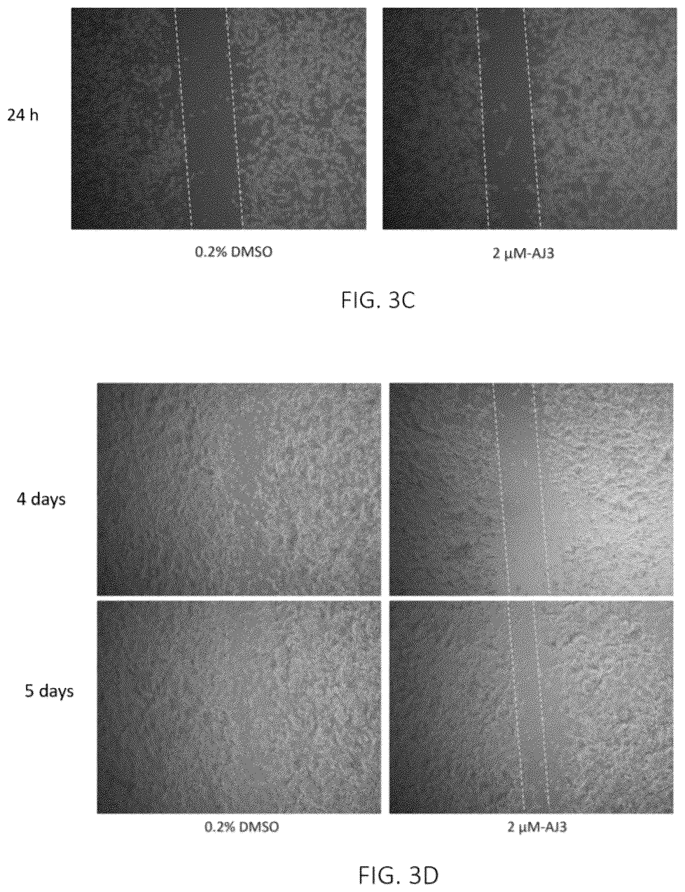

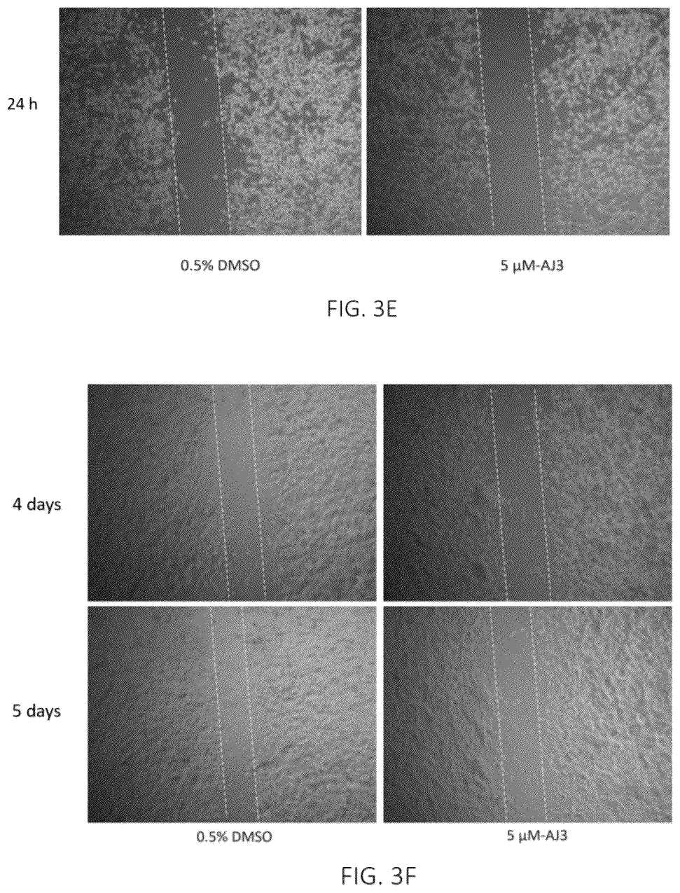

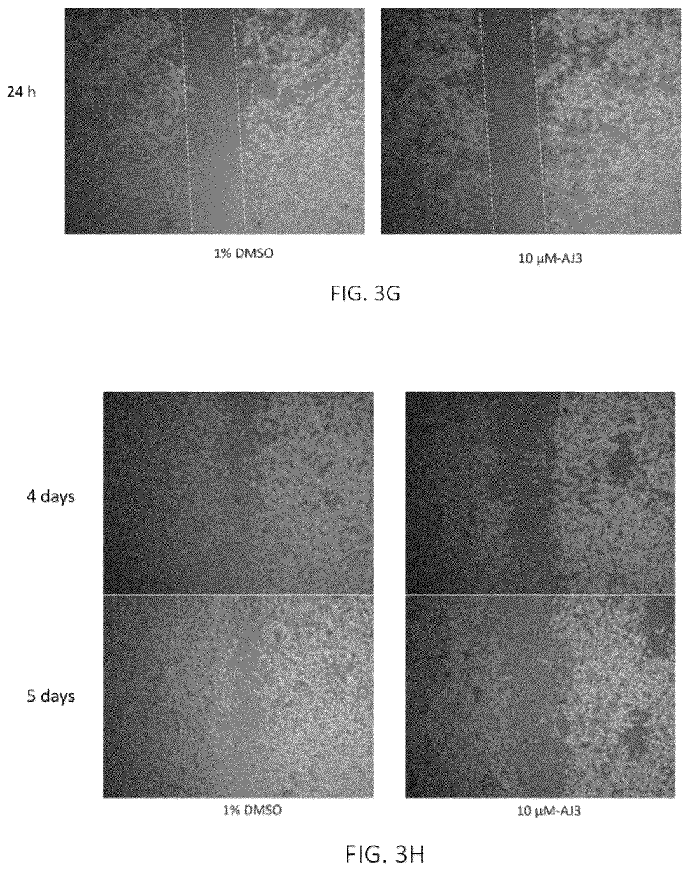

Metastatic evaluation was carried out by a wound healing assay. A wound healing assay measures cell migration of individual cells, cell sheets, and clusters from one location to another. A single mechanical laceration was made on a 12-well plate growing a cancer cells monolayer that was grown overnight, and the cells were allowed to regrow across the inclusion over the span of 48 hours. Results were recorded between the knockdown shRGS2 and control in values of the closure % as the wound healing took place at 24 hour and 48 hour time points (FIG. 3). Results for the RGS2 inhibitor treated cells were recorded as pictures taken at 5 separate time points every 24 hours (FIG. 4A-H).

### Conclusion

Both the shRGS2 knockdown cancer cells and the RGS2 inhibitor treated cancer

cells exhibited a reduction in metastatic potential. The shRGS2 knockdown cancer cells showed a lower amount of closure made across the mechanical laceration after both 24 hours and 48 hours in comparison to the normal RGS2-level LnCap cells. In a similar fashion, the RGS2 inhibitor treated LnCap cells visually showed a slower growth across the mechanical laceration over the course of five days in comparison to its non-treated LnCap control. This loss in metastasis shows the reduction in expressed RGS2, or direct inhibition, leads to a loss of migration ability of cancer cells.

### EXAMPLE 3

Another study was conducted to measure the cytotoxic effect of the RGS2 inhibitor. The growth reduction feature of direct RGS2 inhibitors was examined within the National Cancer Institute single high dose panel established that the drug was cytotoxic across a variety of cancers. Spanning across 60 standard cancer cell lines, this cytotoxicity assessment measured anti-proliferative activity within the following cancer types, with numerous subtypes below them: leukemia, non-small cell lung cancer, colon cancer, central nervous system cancer, melanoma, ovarian cancer, renal cancer, prostate cancer, and breast cancer. In this study, every permutation of the RGS2 inhibitor claimed showed some amount of inhibitory effect against at least two types of cancer, namely renal cancer and leukemia.

The NCI 60 Cell One-Dose Screen is a standardized test carried out by National Cancer Institute, where a compound is tested at an initial single high dose (10⁻⁵ M) across the full NCI 60 cell panel. Once a compound is assessed to be effective against a standard threshold number of cell lines, the compound is progresses to a full five-dose screen to determine anti-proliferative capability. This assay is ran relative to parallel studies with a no-drug control and at time zero number of cells.

### Conclusion

Overall, every RGS2 inhibitor showed some amount of inhibitory effect against at least two types of cancer, namely renal cancer and leukemia. Composition AJ1 (Table 1) exhibited cytotoxic effect against all aforementioned cancers except prostate, ovarian, and colon cancer. Compositions AJ3, AJ4, AJ8, AJ9, and AJ10 (Table 1) have all shown strong cytotoxic effect against the full list of cancers. These results confirm the selective inhibition of the RGS2-G_{alpha-q} protein-protein interaction stops the overgrowth of aggressive human cancers across a wide range of tissues and subtypes.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A method of treating a subject with a cancer, comprising administering to the subject an RGS2 inhibitor in an amount therapeutically effective to treat the cancer.

2. The method of claim 1, wherein the RGS2 inhibitor non-covalently interacts with turn-183 of human RGS2 defined by a first asparagine at residue 183 (“N183”), a second asparagine at residue 184 (“N184”), and a serine at residue 185 (“S185”).

3. The method of claim 2, wherein N183 and N184 interact with a positively charged pharmacophore site and S185 interacts with a hydrogen-bond donor pharmacophore site.

4. The method of claim 1, wherein the RGS2 inhibitor has a structure selected from the group consisting of formulas 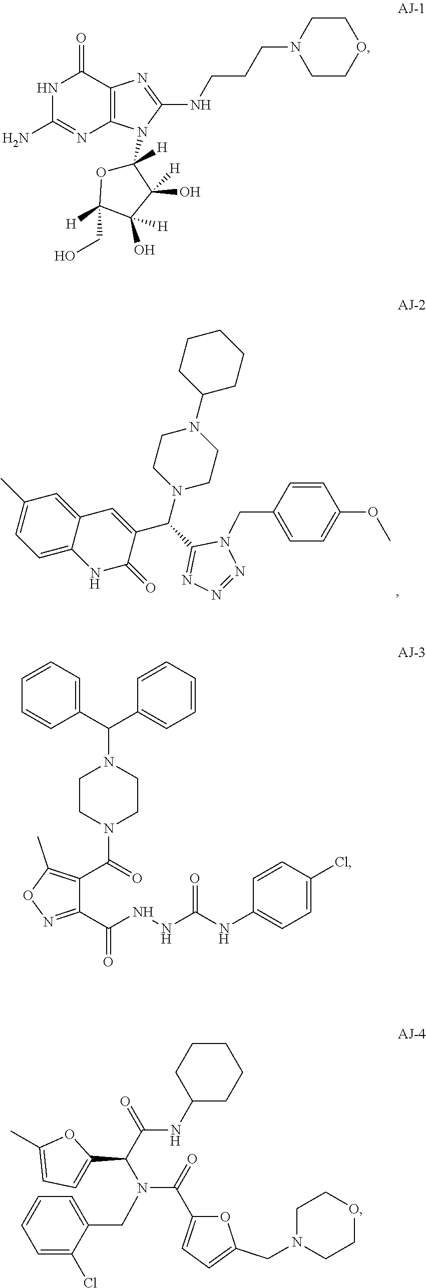 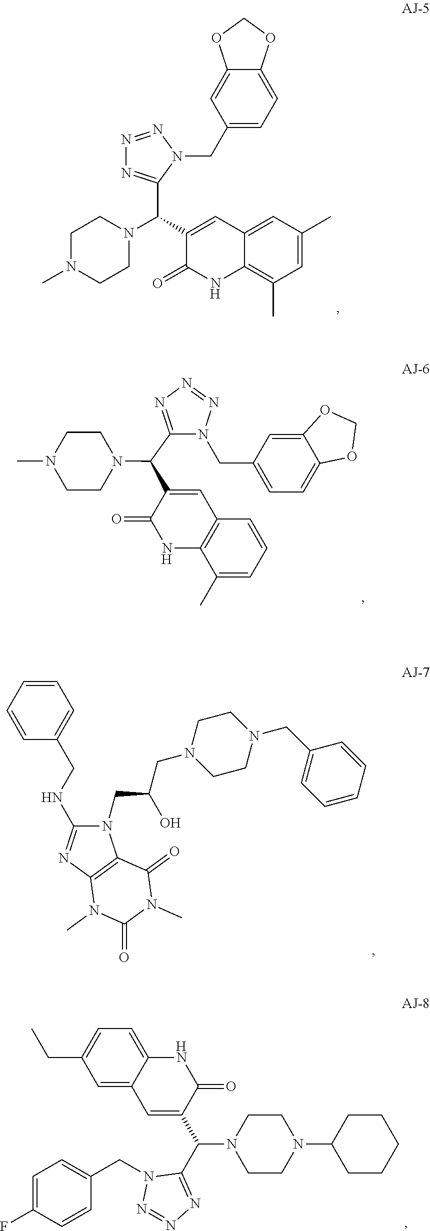 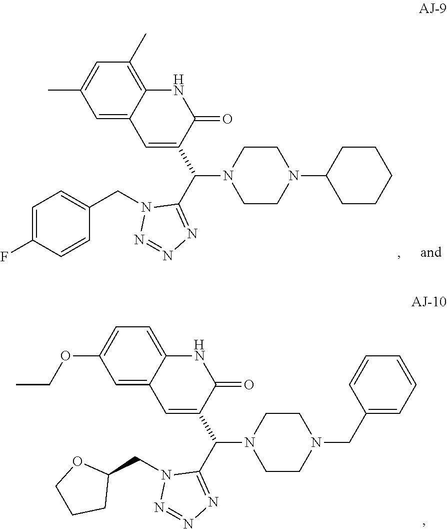
or a pharmaceutically acceptable salt or solvate thereof.

5. The method of claim 1, wherein the cancer is selected from a breast cancer, a prostate cancer, a renal cancer, an ovarian cancer, a melanoma, a colon cancer, a non-small cell lung cancer, a leukemia, a central nervous system cancer.

6. The method of claim 1, wherein the cancer is selected from a solid cancer, a blood cancer, or combinations thereof.

7. The method of claim 1, wherein the RGS2 inhibitor is administered to the subject via oral administration.

8. The method of claim 1, wherein the RGS2 inhibitor is administered to the subject via injection administration.

9. The method of claim 8, wherein the injection administration is selected from the group consisting of intravenous injection, intra-arterial injection, subcutaneous, intramuscular injection, peritoneal injection, intrathecal injection, or combinations thereof.

10. The method of claim 1, wherein the cancer is a central nervous system cancer.

11. The method of claim 10, wherein the RGS2 inhibitor is administered via intrathecal injection.

12. The method of claim 10, wherein the RGS2 inhibitor has a structure with the formula 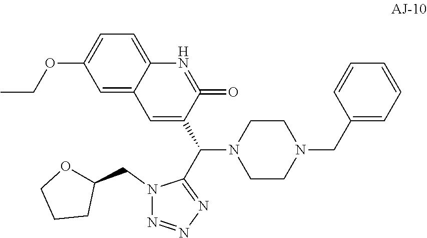
or a pharmaceutically acceptable salt or solvate thereof.

13. A pharmaceutical composition comprising an RGS2 inhibitor in an amount effective to inhibit the interaction between an RGS2 protein and a Gₐₗₚₕₐ protein in a cell of a cancer of a subject.

14. The pharmaceutical composition of claim 13, wherein the Gₐₗₚₕₐ protein is a G_{alpha-q} protein.

15. The pharmaceutical composition of claim 13, wherein the RGS2 inhibitor non-covalently interacts with a first asparagine at residue 183 (“N183”), a second asparagine at residue 184 (“N184”), and a serine at residue 185 (“S185”), wherein N183 and N184 interact with a positively charged pharmacophore site and S185 interacts with a hydrogen-bond donor pharmacophore site.

16. The pharmaceutical composition of claim 13, wherein the RGS2 inhibitor has a structure selected from the group consisting of 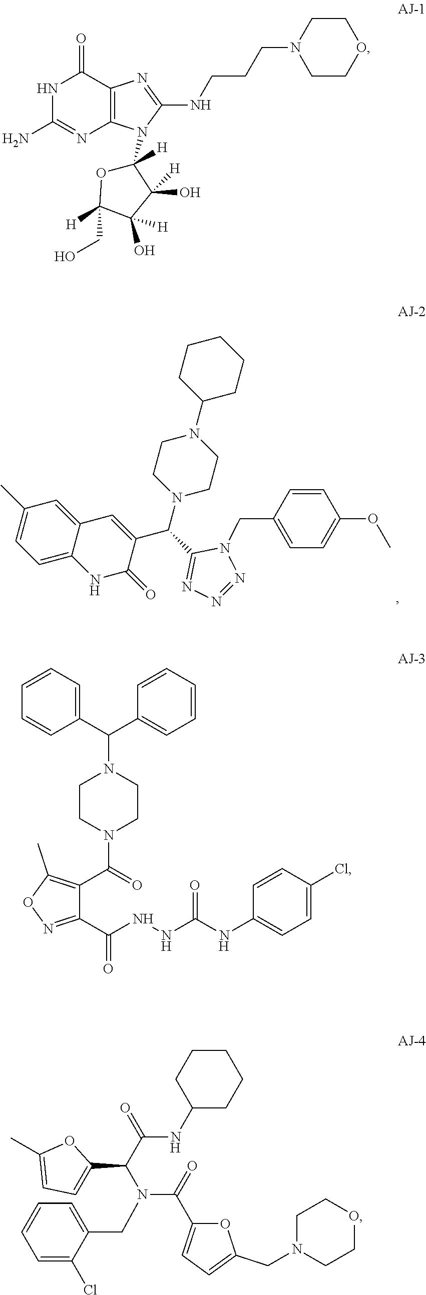 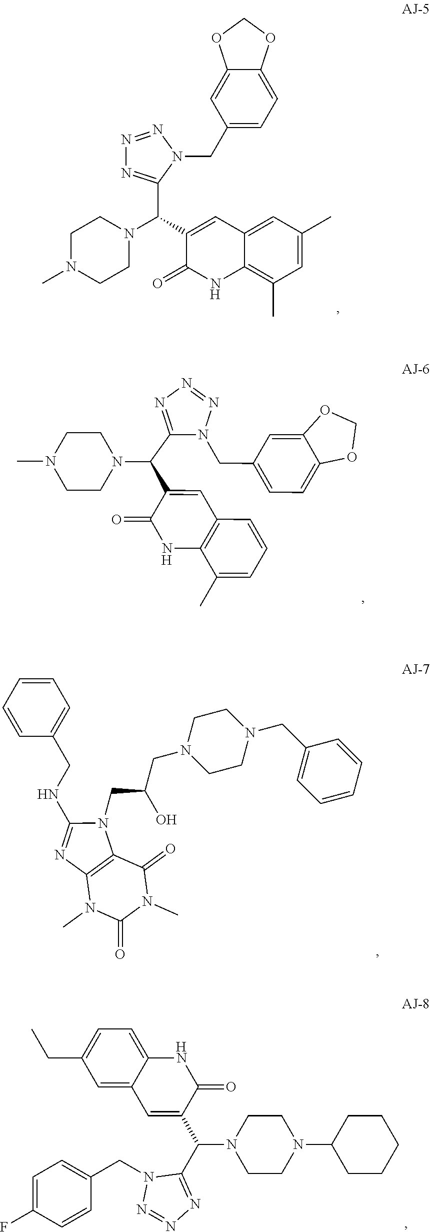 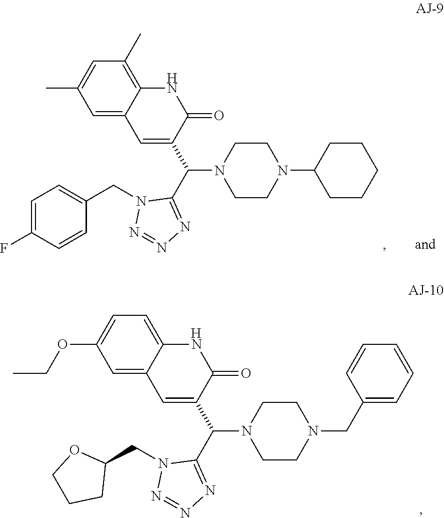
or a pharmaceutically acceptable salt or solvate thereof.

17. The pharmaceutical composition of claim 13, wherein the RGS2 inhibitor is formulated for administration to a subject via oral administration, injection administration, or combinations thereof.

18. A method of inhibiting the interaction between an RGS2 protein and a Gₐₗₚₕₐ protein in a cell comprising administering the cell to an RGS2 inhibitor in an amount capable of non-covalently interacting with the RGS2 protein.

19. The method of claim 18, wherein the RGS2 inhibitor non-covalently interacts with a first asparagine at residue 183 (“N183”), a second asparagine at residue 184 (“N184”), and a serine at residue 185 (“S185”), wherein N183 and N184 interact with a positively charged pharmacophore site and S185 interacts with a hydrogen-bond donor pharmacophore site.

20. The method of claim 18, the RGS2 inhibitor has a structure selected from the group consisting of 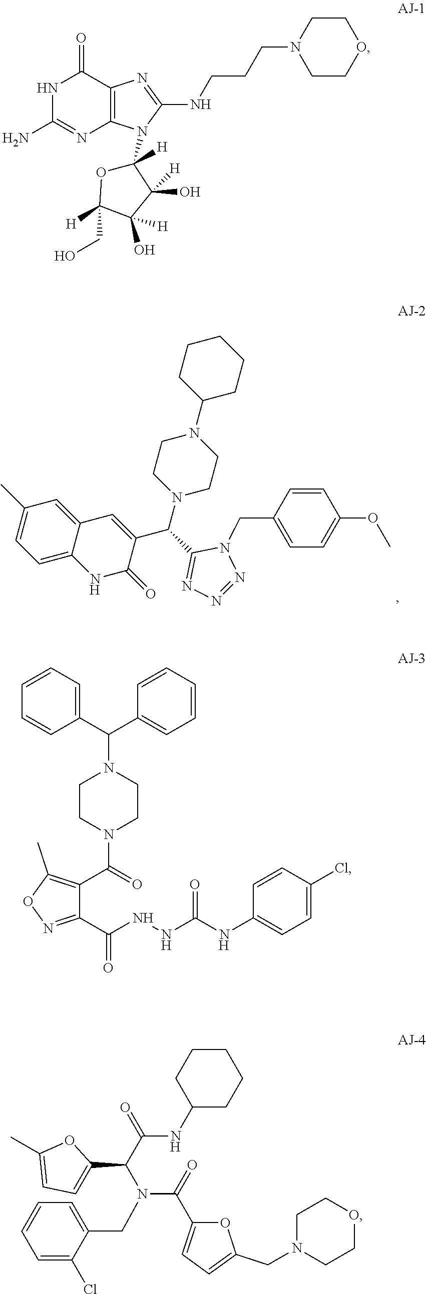 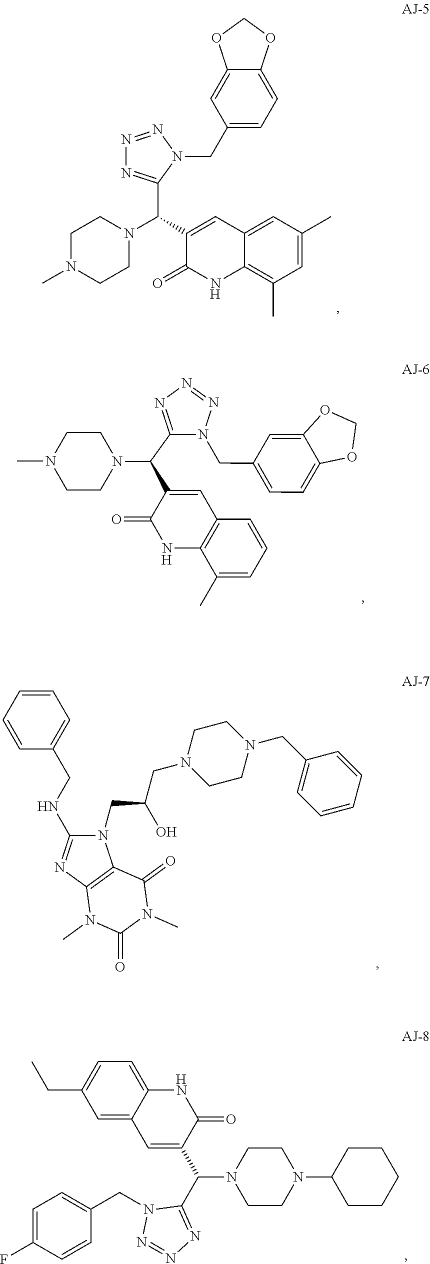 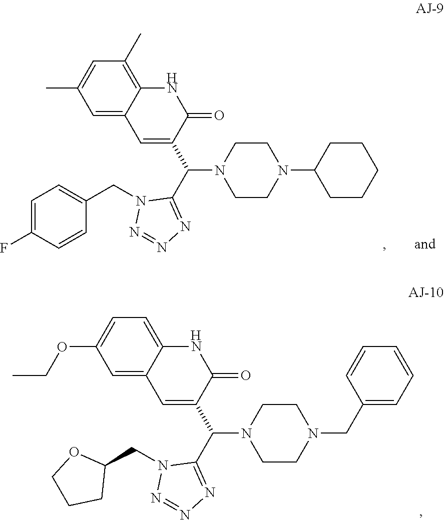
or a pharmaceutically acceptable salt or solvate thereof.
